(19) United States
(12) Patent Application Publication
(10) Pub. No.: US 2014/0296968 A1
(43) Pub. Date: Oct. 2, 2014

(54) IMPLANTABLE VASCULAR STENT

(71) Applicants: Christian Bader, Wannwell (DE); Boris Behnisch, Tuebingen (DE)

(72) Inventors: Christian Bader, Wannwell (DE); Boris Behnisch, Tuebingen (DE)

(73) Assignee: Translumina GmbH, Hechingen (DE)

(21) Appl. No.: 14/131,664

(22) PCT Filed: Jul. 5, 2012
(86) PCT No.: PCT/EP2012/063108

§ 371 Date: Mar. 12, 2014

(30) Foreign Application Priority Data
Jul. 12, 2011 (DE) 10 2011 107 109.5

(51) Int. Cl. A61F 2/82

(52) U.S. Cl. CPC A61F 2/82
USPC 623/1.46

(57) ABSTRACT

A stent for implantation in a vessel is described that is radially expandable, and when expanded in a vessel, lies against the inner wall thereof. The stent is provided with a coating applied to its surface and composed of a bioresorbable oligomeric binding agent and of an active substance (FIG. 1).

The present invention concerns a radially expandable stent for implantation in a vessel, which stent has at least one metal surface and, when expanded in the vessel, lies against the inner surface of the vessel, said stent being provided with a mixture of a bioresorbable lactic/glycolic-acid-based binding agent and an active substance, said mixture applied to its surface.

This type of stent is known from EP 2 189 169 A1.

Stents within the meaning of the present invention are intravascular implants, which vessel supports are also referred to as stents. Such stents are radially expandable endoprostheses that are implanted transluminally in vessels such as blood vessels, the esophagus, trachea, and intestinal tract and the like, and then radially expanded so that they lie against the inner wall of the vessel.

For example, stents are used in the vascular system to reinforce blood vessels and/or prevent restenosis following previous angioplasty. They can be self-expanding or be actively expanded by a radial force exerted from inside, e.g. when mounted on a balloon.

The term “radially expanding stent” used in the present invention is therefore understood to include both self-expanding and actively expandable stents.

In this case, the stents show a hollow cylindrical body having a wall composed of interconnected struts and branches that form a radially permeable and flexible structure. In a frequently preferred embodiment, the hollow cylindrical body comprises ring-shaped segments composed of self-closed branches running in a zigzag pattern that form loops lying circumferentially adjacent to one another and having proximal and distal ends, so that each segment has an undulating meander-like structure along its circumference. The individual segments are connected to one another by short struts. On expansion, the distance between the proximal and distal ends of each segment becomes somewhat shorter, but the zigzag structure remains intact.

When expanded, the external diameter of said body corresponds roughly to the internal diameter of the vessel to be supported, the stent lying against the wall of said vessel by the radial force exerted by its flexible and elastic structure. In a longitudinal direction, the body of the stent is open in order to allow the passage of media or substances to be transported in the supported vessel.

As a rule, these stents are placed in the body using so-called introduction systems and released at the target site, for which purpose they are loaded onto a catheter—i.e., crimped onto its balloons—which is advanced through the relevant vessels according to the so-called Seldinger technique using guide wires running through the inner lumen of the catheter.

When the delivery site in the relevant vessel has been reached, the stent is expanded, and thus lies with its surface against the vessel wall.

Specifically, in another known configuration, the surface is coated abluminally with active substances, more specifically drugs, or drug reservoirs are provided within the structure of the stent and/or microporous struts and branches are used for temporary storage of the drug. The drug is then locally delivered to the wall of the vessel, e.g. in order to prevent restenosis due to proliferation of the surrounding tissue.

By this method, stents that are coated in this manner or provided with a reservoir allow the targeted on-site delivery, as it were, of active substances to the surrounding tissue. The coating of stents, i.e. vascular prostheses, with active substances is also advantageous because this improves the biocompatibility of the implant, which e.g. makes it possible to prevent the occurrence of thrombosis with surfaces that come into contact with the blood.

This supply of drugs to the vessel wall is of particular importance in stents having a bare metal surface, referred to as bare-metal stents (BMS), as such stents show a relatively high restenosis rate of approximately 30%. This is attributable to vessel damage triggered by implantation that results in the proliferation of smooth muscle cells.

Therefore, in order to prevent and/or alleviate proliferation of smooth muscle cells, stents are always coated with cystostatic or cytotoxic drugs such as rapamycin or paclitaxel. Such stents are referred to as drug-eluting stents (DES).

For this purpose, the drugs are attached to the surface of the stent using a binding agent, usually a polymeric binding agent, or incorporated into a polymer matrix on the surface of the stent. After implantation of the stent, the drug is delivered to the vessel wall via diffusion from the polymer. After successful drug delivery, the polymer permanently remains on the surface of the stent.

The residual polymer may lead to inflammations and sensitization reactions, with the result that in-grow of the stent is not as favorable as in a comparable BMS. In the worst-case scenario, not grown-in stent struts result in late stent thrombosis. Accordingly, the lower restenosis rate compared to BMS must be weighed against the subsequent increased incidence of late stent thrombosis.

It is therefore desired to provide a DES that shows both favorable antirestenotic properties and good grow-in performance, and thus a lower risk of late stent restenosis. This requirement could be met using polymer-free DES. Because production thereof is difficult from a technical standpoint, however, it has already been suggested that bioresorbable binding agents should be used with DES.

After delivery of the drug, stents comprising such biodegradable polymers are converted into BMS due to degradation of the polymer components and thus combine the favorable safety properties of BMS with the favorable antirestenotic properties of a DES.

In such cases, the polymers mainly used are various lactic and/or glycolic-acid-based polyesters that degrade inside the body without leaving any residue. A known technique is the use of PLA, PLLA, and PLGA with various monomer ratios. These polymers sometimes differ markedly from one another in their mechanical and chemical properties.

A stent having a bioresorbable PLGA-based binding agent is described in the publication of Falotico et al., “NEVO™: a new generation of sirolimus-eluting coronary stent,” in EuroIntervention Supplement (2009), Vol. 5 (Supplement 5), F88-F93.

The known stent is provided with reservoirs evenly distributed over its structure and composed of through openings in the stent material. These openings are closed with a floor of PLGA either toward the interior or the exterior, with the monomer ratio of lactic to glycolic acid in this case being 75:25, so that the polymer degrades according to this publication within 4 to 5 months.

The blind hole formed in this manner is filled with a mixture of PLGA and the drug sirolimus in a 1.2:1 ratio. The delayed release of the drug either outward or inward takes place both by diffusion of the drug from the polymer matrix and by degradation of the polymer matrix in the reservoir.

According to this publication, 80% of the drug is delivered within 30 days, and it is stated that 100% of the drug has been released after 90 days. In addition, this known stent is said to be completely converted to a BMS after about three months. The publication does not disclose whether all of the binding agent was degraded or there was still residue left in the reservoir openings.

While this known stent appears to meet the essential requirements for favorable antirestenotic properties and good in-grow performance, it does show a considerable number of drawbacks.

On the one hand, filling of the reservoir is technically complex and time-consuming, as for abluminal delivery of the drug, it is first necessary to place the PLGA floor in each individual opening and then fill the resulting blind holes with the mixture of the polymer and the drug. If luminal delivery is also required, a portion of the reservoir must be first be filled with the mixture and then capped with a PLGA. For two time, two production steps are therefore required.

Furthermore, the monomer ratio of 75:25 by no means ensures that the PLGA floor or cover will be completely degraded after three months, with the result that the polymer is still being released into the tissue several weeks after complete delivery of the drug, which can lead to problems with inflammation and sensitization.

The stent known from aforementioned EP 2 189 169 A1 is composed of a magnesium alloy and is therefore completely biodegradable by itself. The binding agent contains the polymer polylactide co-glycolide and is intended primarily to delay degradation of the magnesium; it can also serve as a matrix for delivering the active substance.

US 2010/0042206 A1 also describes a completely biodegradable stent composed of a magnesium alloy that may be provided with a coating of various lactic/glycolic acid-based polymers, with said polymers serving as the matrix for delivering active substances. Delivery of the active substance and degradation of the matrix are to take place in parallel over a period of 1 to 12 months.

WO 2004/087214 A1 also describes a stent that may be provided with a coating of lactic/glycolic-acid-based polymers that can serve as a matrix for the delivery of active substances. This known stent has a similar structure to the stent known from the aforementioned publication of Falotico et al. It shows pores in which the active substance is held in place by PLGA until it is delivered.

US 2008/0169582 describes the production of stents composed entirely of biodegradable long-chain polymers. The active substance is either incorporated into the stent material or embedded in a matrix composed of an additional polymer layer. The polymers may be based on lactic and glycolic acid.

In view of the above, the object underlying the present invention is to provide a stent of the kind mentioned at the outset in which the drawbacks known from prior art are avoided. Specifically, production of the stent is to be simplified, and the delivery kinetics of the active substance and degradation of the binding agent are to be improved.

With the stent mentioned at the outset, this object is achieved in that the bioresorbable binding agent is an oligomer based on lactic and glycolic acid and having a chain length that is sufficiently short to allow the oligomer to be biologically degraded within about 6 weeks while implanted.

As, according to the invention, the mixture of oligomeric binding agent and active substance is applied to the surface as a coating, no additional binding agent areas such as the covers or floors with known stents are required in order to keep the mixture intact over long periods. The oligomeric binding agent can therefore be configured in such a way that it degrades at roughly the same rate as that at which the active substance is released.

For this purpose, the invention uses as a binding agent an oligomer, i.e. a substance having a degree of polymerization that is below 100 according to the conventional definition. Oligomers used within the scope of the invention show a degree of polymerization that is approximately lower than 50.

In this case, it is particularly advantageous that washing or dissolving the drug from the binding agent matrix and degradation of this matrix itself can take place in parallel, so that after complete delivery of the drug, there is no residual binding agent remaining on the stent.

The feature of allowing, according to the invention, to adjust a corresponding degradation rate makes it possible to reduce the time the binding agent is present on the stent to the shortest period possible, thus largely avoiding the drawbacks described above of inflammation and sensitization reactions.

Moreover, since the coating is directly applied to the surface of the stent, the need for the time-consuming production steps required for the stent known from the publication by Falotico et al is obviated.

Another advantage is that the surface coating according to the invention, which preferably lies in closed form on the surface, provides uniform abluminal release of the drug, a characteristic that, despite a possibly large number of reservoirs, is not guaranteed by the stent known from the publication of Falotico et al.

The new stent thus not only provides excellent drug delivery kinetics and rapid degradation of the oligomeric binding agent, but also allows drug delivery that is uniformly distributed over the surface.

The objective underlying the invention is by this fully achieved.

In this connection, it is preferred that the bioresorbable binding agent is an Oligo(D,L-lactate-co-glycolate) having a chain length sufficiently short to allow the agent to be degraded within approximately 6 weeks while implanted, and the oligomer should preferably have a molecular weight (M_{η}) of between 1,000 to 4,000 Daltons, and preferably of approximately 3,000 Daltons, and preferably a molecular ratio of lactic to glycolic acid in the oligomer of approximately 1:1.

This coating has been found to be particularly effective in experiments conducted by the inventors to date.

To the knowledge of the inventors, neither the use of oligomers based on lactic and glycolic acid nor of Oligo(D,L-lactate-co-glycolate) as a binding agent has been proposed in the prior art. For use as binding agent have either unspecifically been suggested long-chain polymeric PLGA, i.e. poly(lactic acid-co-glycolic acid), namely a polymer produced by polycondensation of the two acids, or specifically poly(lactide-co-glycolide), i.e. a polymer of different crystallinity that is produced by ring-opening polymerization of the two diesters.

The inventors have now recognized that binding agents composed of short-chain oligomers, particularly those produced from the salts of lactic and glycolic acid, are particularly well-suited as a matrix for active substances, because they degrade under physiological conditions within 6 weeks. This period corresponds to the period within which the active substance is to be delivered.

It is known from the publication of Falotico et al. discussed above that the degradation rate of PLGA, i.e. of polymers, is affected by the monomer ratio of lactic to glycolic acid and the molecular weight of the polyester, expressed here via inherent viscosity. According to this publication, the most rapid degradation rate is achieved at a monomer ratio of 50:50, with this degradation rate increasing with decreasing viscosity.

For polymers with a monomer ratio of 50:50, the degradation times are said to be 1-2 weeks, 3-4 weeks, or 1-2 months, depending on viscosity. There is no mention of the conditions under which these degradation times were determined. Measurements conducted by the inventors of the present invention show distinctly longer degradation times than those given in the publication of Falotico et al.

The known stent uses a monomer ratio of 75 25, with the degradation time said to be approximately 4-5 months. In the known stent, this polymer is used in such a manner that it does not come into mechanical contact with the vessel wall during implantation.

After 3 months, when the active substance has been completely delivered, in the known stent residual binding agent is therefore still present in the reservoir openings and is only degraded in the following 4-8 weeks. This is also reasonable, as the binding agent of said stent is used among other purposes to define and limit, respectively, the direction of drug delivery, such that the binding agent shall not fully degrade until 100% of the active substance has been delivered.

The inventors of the present invention have now recognized that oligomers based on lactic and glycolic acid, particularly Oligo(D,L-lactate-co-glycolate), are particularly well-suited for use in applying a preferably closed coating to the surface of a stent, and that by incorporation of an active substance into the binding agent matrix, it becomes possible by the oligomers used according to the invention to select delivery kinetics in such a manner that the binding agent has already been degraded after complete delivery of the active substance, with only a BMS remaining.

It was not to be expected that this coating with the oligomers used according to the invention nevertheless provides sufficient mechanical stability, such that the stent after application of the coating can be crimped onto a balloon and dilated after being inserted into the vessel without causing the coating to unravel, chip off, or agglutinate.

Specifically, according to the findings of the inventors, the oligomer provides good cohesion within the coating and good adhesion between the coating and the surface of the stent.

Oligomers having the degradation times provided according to the invention are not shown in the publication by Falotico et al.

In particular, when the oligomer is mixed with rapamycin or a rapamycin derivative, preferably in a weight ratio of between 3:1 to 1:3, and more preferably of approximately 1:1, this provides a stable coating that degrades completely and without residue within approximately 6 weeks.

Experiments with pure rapamycin coatings have shown that this is dissolved in vitro from the stent surface in 4-6 weeks, depending on the thickness of the layer.

Accordingly, the new coating is advantageous in that after complete dissolution and/or washing out of the rapamycin components, no binding agent residue, which could adversely affect safety properties, is found on the stent surface.

This release takes place not only due to diffusion or washing out of the active substance, but also due to biodegrading of the binding agent.

The use of oligomers and rapamycin in a weight ratio of 1:1 is advantageous in certain applications, but other weight ratios may also be used within the scope of the present invention.

The coating is preferably applied to a stent with a microporous or rough surface, with the metal of the stent preferably being a stainless steel. In this case, the stent may be composed completely of the metal, preferably the stainless steel, or have a metal surface, with said surface being directed both toward the inner wall of the vessel and, if applicable, the lumen of the vessel.

After complete delivery of the active substance and the accompanying complete degradation of the binding agent, i.e. the matrix, the remaining surface is a pure and microporous metal surface that shows the favorable grow-in properties of a BMS. Nevertheless, the new stent also shows the antirestenotic performance of a DES.

The use of the oligomeric binding agent thus makes it possible for the first time to achieve rapid and complete grow-in of a DES, which is generally associated with a high degree of safety, e.g. in coronary stents.

Stents having a microporous surface are known from the prior art; see for example DE 102 00 387 A1. The surface of the known stent is provided with pores in which drugs and polymers are kept in reserve. However, it is also possible to configure the stent with a rough surface provided with tiny pores.

According to the findings of the inventors of the present invention, particularly because of the film-forming properties of the oligomers used according to the invention, which are clearly different from the film-forming properties of polymers, the present invention makes it possible to produce stable coatings on expandable stents, which are particularly stable if the stent has a microporous or rough surface.

Furthermore, it is preferred if the surface is provided with a closed coating composed of the bioresorbable binding agent and the active substance.

Within the scope of the present invention, a “closed coating” is understood to refer to a coating that covers at least the entire abluminal surface of the stent and shows no chipping off over larger areas, even during crimping and subsequent dilation of the stent.

Further advantages can be seen in the description and the attached drawing.

It is to be understood that the aforementioned features and those to be discussed below may be used not only in the respectively indicated combination, but also in other combinations or individually without departing from the scope of the present invention.

An embodiment of the invention is presented in the attached drawing and will be discussed in more detail in the following description. In the figures:

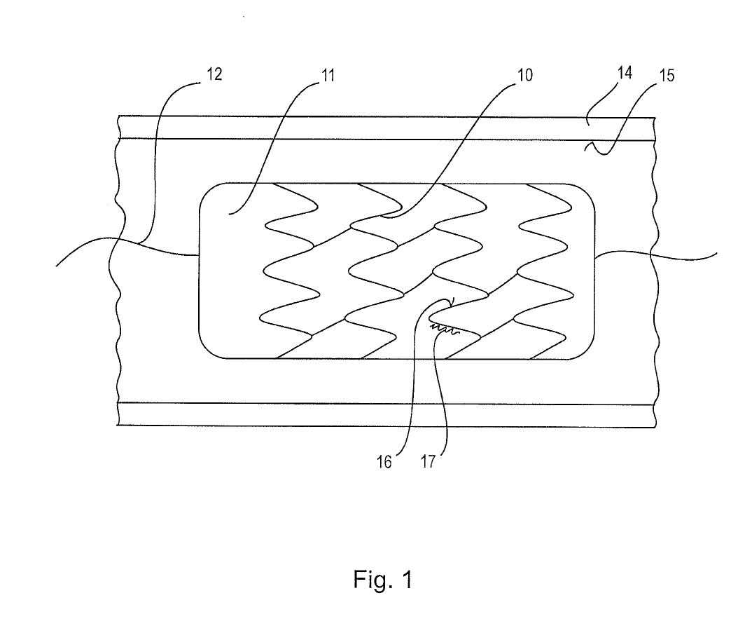

FIG. 1: shows a schematic, sectional side view of a stent having the coating according to the invention, after insertion into a vessel and before dilation;

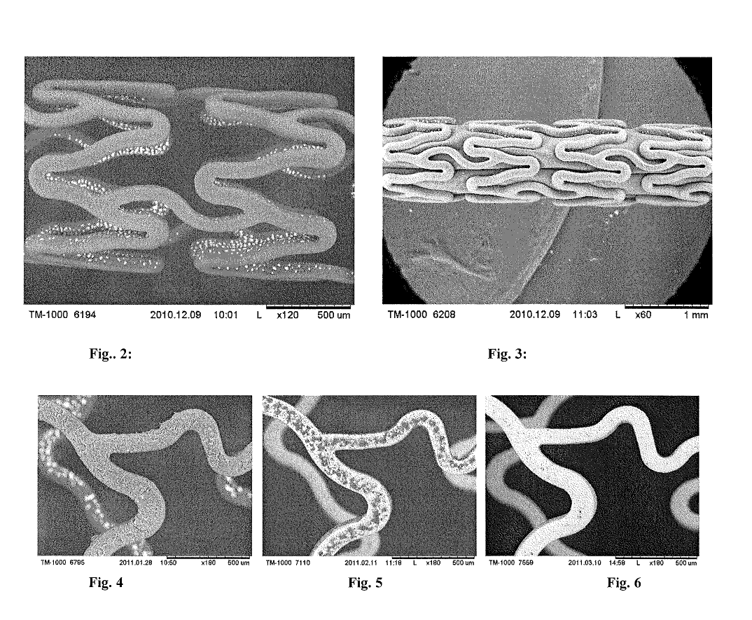

FIG. 2: shows a photograph of a stent surface coated according to the invention and not yet crimped;

FIG. 3: a photograph as in FIG. 2, but after crimping;

FIG. 4: a presentation as in FIG. 3, but in enlarged view and after dilation;

FIG. 5: a presentation as in FIG. 4, but after 2 weeks of contact with an isotonic saline solution at 37° C.; and

FIG. 6: a presentation as in FIG. 4, but after 6 weeks of contact with the isotonic saline solution.

FIG. 1 shows a schematic side view of a stent 10 that is crimped onto a balloon 11. The balloon 11 has been advanced by means of a guide wire 12 into a vessel 14, and it comes into contact with the wall 15 of the vessel after expansion of said balloon 11.

The stent 10 shows a surface 16 to which a coating schematically indicated by 17 has been applied, with said coating containing a binding agent and rapamycin as an active substance in a ratio by weight of 1:1. The coating thickness is approximately 7 to 10 μm, and the coating solution, from which the coating on the surface was deposited, was approximately 2% (=10 mg/mL rapamycin and 10 mg/mL binding agent).

The surface 16 is microporous, i.e. it shows pores in the diameter range of 1 to 5 μm. The stent 10 corresponds e.g. to the stent known from DE 102 00 387 A1. However, it may also have a roughened surface 16.

The binding agent is an oligoester containing the monomers lactic acid and glycolic acid in a ratio of 1:1. The oligomer (B2) has a molecular weight determined based on limit viscosity of approximately 3,000 (B2) Daltons and a degree of polymerization of approximately 45. It is commercially available from the company Evonik under the brand name Resomer® Condensate 50:50 Mₙ 2300.

For comparison purposes, a polymer (B1) having a degree of polymerization of approximately 142 and a molecular weight of approximately 10,000 Daltons was studied. PLGA B1 is sold by Evonik as Resomer® RG 502 H.

This coating degrades during contact with tissue after 2 weeks by 20% (B1) and 80% (B2), respectively, and after 6 weeks by 90% (B1) and >99% (B2), respectively.

The best values were found for B2, this coating degrades after 6 weeks by 100%.

In principle, both coatings are suitable for use on stents, as they show favorable mechanical stability.

However, only B2 yielded mechanically satisfactory results, with a degradation rate that corresponds to the release of rapamycin, which takes approximately 4 to 6 weeks in vivo.

FIGS. 2 and 3 show the stent coated with B2 immediately after coating and after crimping.

FIGS. 4 through 6 show a magnified electron microscopic photograph of the stent of FIG. 2 after crimping and subsequent dilation, immediately after coating (FIG. 4), and after storage for 2 weeks (FIG. 5) and 6 weeks (FIG. 6) in isotonic saline solution at 37° C.

It should be noted that the mechanical quality of the surface coating is not adversely affected by crimping, and that the coating has completely disappeared after 6 weeks.

1-10. (canceled)

11. A radially expandable stent for implantation in a vessel, said vessel having an inner wall,
said stent having at least one metal surface that lies against said inner wall when said stent is expanded in said vessel,
a coating comprising a mixture of a bioresorbable binding agent and of an active substance being applied to said surface, and
the bioresorbable binding agent being an oligomer based on lactic and glycolic acid and having a chain length that is short enough for the oligomer to be biologically degraded within approximately 6 weeks when the stent is implanted into the vessel.

12. The stent of claim 11, wherein the oligomer is an Oligo(D,L-lactate-co-glycolate).

13. The stent of claim 11, wherein the oligomer has a molecular weight (M_{η}) of between 1,000 to 4,000 Daltons.

14. The stent of claim 12, wherein the oligomer has a molecular weight (M_{η}) of between 1,000 to 4,000 Daltons.

15. The stent of claim 13, wherein the oligomer has a molecular weight (M_{η}) of approximately 3,000 Daltons.

16. The stent of claim 14, wherein the oligomer has a molecular weight (M_{η}) of approximately 3,000 Daltons.

17. The stent of claim 11, wherein the molecular ratio of lactic to glycolic acid in the oligomer is approximately 1:1.

18. The stent of claim 11, wherein the binding agent and the active substance are present in the coating at a ratio by weight of between 3:1 to 1:3.

19. The stent of claim 18, wherein the binding agent and the active substance are present in the coating at a ratio by weight of approximately 1:1.

20. The stent of claim 11, wherein the active substance comprises a substance selected from rapamycin and a rapamycin derivative.

21. The stent of claim 11, wherein the metal surface is selected from a microporous surface and a rough surface.

22. The stent claim 11, wherein the metal surface is provided with a closed coating composed of the bioresorbable binding agent and the active substance.

23. The stent of claim 11, wherein the metal of the metal surface is a stainless steel.

24. The stent of claim 11 which is fabricated from metal.

25. The stent of claim 11 which is fabricated from a stainless steel.

26. A radially expandable stent for implantation in a vessel, said vessel having an inner wall,
said stent having at least one metal surface,
a coating being provided on said metal surface, said coating comprising a mixture of a bioresorbable binding agent and an active substance, and
the bioresorbable binding agent being Oligo(D,L-lactate-co-glycolate) having a molecular weight (M_{η}) of between 1,000 to 4,000 Daltons.

27. The stent of claim 26, wherein the molecular ratio of lactic to glycolic acid in the Oligo(D,L-lactate-co-glycolate) is approximately 1:1.

28. The stent of claim 26, wherein the binding agent and the active substance are present in the coating at a ratio by weight of between 3:1 to 1:3.

29. The stent of claim 26, wherein the metal surface is selected from a microporous surface and a rough surface.

30. The stent claim 26, wherein the metal surface is provided with a closed coating composed of the bioresorbable binding agent and the active substance.

31. The stent of claim 26, wherein the metal of the metal surface is a stainless steel.

32. The stent of claim 26 which is fabricated from metal.

33. The stent of claim 26 which is fabricated from a stainless steel.

34. A radially expandable stent for implantation in a vessel, said vessel having an inner wall,
said stent being fabricated from metal and having at least one surface that bears against said inner wall when said stent is expanded in said vessel,
a closed coating being provided on said surface, said closed coating comprising a mixture of a bioresorbable binding agent and of an active substance, and
the bioresorbable binding agent being Oligo(D,L-lactate-co-glycolate) having a molecular weight (M_{η}) of between 1,000 to 4,000 Daltons.
